(19) United States
(12) Patent Application Publication
(10) Pub. No.: US 2010/0189767 A1
(43) Pub. Date: Jul. 29, 2010

(54) PROBIOTIC COMPOSITIONS AND METHODS OF MAKING SAME

(76) Inventors: Eyal Shimoni, Haifa (IL); Ory Ramon, Sarid (IL); David Semyonov, Ma'alot (IL); Saul Koder, Misgav (IL); Uri Korkin, Binyamina (IL)

Correspondence Address: WINSTON & STRAWN LLP;PATENT DEPARTMENT, 1700 K STREET, N.W., WASHINGTON, DC, 20006, US

(21) Appl. No.: 12/442,063

(22) PCT Filed: Sep. 18, 2007
(86) PCT No.: PCT/IL07/01143

§ 371 Date: Mar. 16, 2010

Related U.S. Application Data
(60) Provisional application No. 60/845,536, filed on Sep. 19, 2006.

(51) Int. Cl. A61K 9/50; A61K 35/74; A61K 9/00; A61K 9/14; A61P 1/00

(52) U.S. Cl. 424/439; 424/490; 424/93.45; 424/93.4; 424/488

(57) ABSTRACT

The present invention provides solid compositions of bioactive agents, in particular of probiotic microorganisms. Furthermore, the present invention provides methods for preparing these compositions. The methods include microencapsulating live microorganisms to produce a dry formulation and optionally coating the microcapsules while retaining to a significant extent the viability of the microorganisms.

### FIELD OF THE INVENTION

The present invention provides solid compositions comprising bioactive agents, in particular probiotic microorganisms. Furthermore, the present invention provides methods for preparing compositions of the invention, comprising the step of microencapsulating live microorganisms to produce a dry formulation and optionally coating the microcapsules, while retaining to a significant extent the viability of the microorganisms.

### BACKGROUND OF THE INVENTION

In the fast-growing trade of functional foods and nutraceuticals, bioactive ingredients such as probiotics are one of the fastest growing segments of the market. To date, commercially available probiotics are either liquid dietary supplements added only to liquid dairy foods, or dry powder within capsules for oral administration. However, the viability of probiotic microorganisms within dry formulations known in the art, including acidophilus and the like, is extremely low, as low as 1%. Moreover, the viable probiotics in the known dry compositions decreases significantly under the conditions involved during industrial processing, namely, extreme temperatures and exposure to oxygen. In addition, the viability of probiotics in known dry compositions is not maintained during passage in the GI tract.

Probiotics are microorganisms which, when administered in adequate numbers, confer a health benefit upon the host. Several approaches have been investigated for improving the technological and therapeutic performance of probiotics, including strain selection and probiotic stabilization during spray drying and/or freeze drying and gastric transit, as described in Ross et al. (Journal of Applied Microbiology, 98:1410-1417, 2005) and references cited therein.

U.S. Pat. No. 5,897,897 teaches the use of glassy matrices of carbohydrate, in particular made of a modified starch and of a polyhydric alcohol such as propylene glycol or glycerin, prepared by the use of aqueous plasticizers with melt extrusion, to encapsulate flavoring agents and other substances sensitive to environmental effects such as oxidation. This disclosure relates only to pharmaceutical compounds and is not disclosed or suggested as useful for probiotic organisms or other bacteria.

U.S. Pat. No. 6,592,863 discloses a nutritional supplement comprising probiotics, maltodextrin and, optionally, resistant starch. However, this patent does not disclose coating or encapsulating the probiotic microorganisms in the nutritional supplement in a matrix, and does not disclose enhanced viability or enhanced stability in the GI tract or in extreme conditions such as high temperature and oxidation.

Compositions of trehalose and borate confer long-term stability of enzymes, as disclosed for example in Miller, D. P., et al. (Pharmaceutical Research, 15(8):1215-1221, 1998). Attempts to freeze-dry probiotic microorganisms in a composition comprising trehalose (20-32%), resulted in improved shelf life of the encapsulated probiotic microorganisms (Conrad et al, Cryobiology, 41:17-24, 2000); De Giulio et al, World Journal of Microbiology & Biotechnology, 21:739-746, 2005). None of these references, however, utilized maltodextrin as a glassy matrix to preserve the viability of probiotic microorganisms.

In the pharmaceuticals industry to date, food-grade enterocoatings have been used for coating foods and drugs, enabling their controlled release after the passage through the stomach. However, their use for coating encapsulated probiotic compositions has not been disclosed or even suggested.

Probiotics remain challengingly devoid of dry forms, encapsulated or otherwise prepared, wherein viability is significantly preserved, due to their sensitivity to processing and storage conditions. There is an unmet need for dry forms and probiotic compositions capable of maintaining high viability and stability of the microorganisms under industrial and physiological conditions, such as the conditions in the GI tract, extreme temperatures and exposure to oxygen.

### SUMMARY OF THE INVENTION

The present invention relates generally to compositions and methods for incorporating sensitive bioactive agents into a glassy matrix. More particularly, the present invention provides solid compositions comprising probiotic microorganisms encapsulated with a matrix comprising a combination of one or more disaccharide or oligosaccharide sugars (e.g. trehalose) and one or more dextrins (e.g. maltodextrin). Compositions of the invention are, in another embodiment, particularly stable at high temperatures such as the temperature used during drying processes as well as during storage. According to certain embodiments, the microcapsules are further coated by food-grade enteric coating materials. In other embodiments, the coating materials delay release of microorganisms until the intestine. The present invention further provides a method for evaluating the viability of the probiotic microorganisms within the microcapsules.

Surprisingly, the viability of probiotic microorganisms within compositions of the invention is exceptionally high, in some cases as high as 70%. Usually, with compositions known in the art, microbial survival during processing, especially during drying, is within the range of 1-5%; a further drop in viability is observed during storage.

According to one embodiment, the present invention provides a solid composition comprising microcapsules consisting of probiotic microorganisms and a carbohydrate matrix comprising at least one dextrin and at least one cytoprotective disaccharide or oligosaccharide.

According to another embodiment, the microcapsules are coated with a food-grade coating. According to yet another embodiment, the coating comprises one or more compounds selected from the group consisting of: wax, shellac, resistant starch, zein protein, ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate. According to yet another embodiment, the microcapsules further comprise a carrier. According to yet another embodiment, the carrier is selected from a group consisting of: microcrystalline cellulose, spray dried resistant starch and spray dried starch.

According to another aspect, the present invention provides a method for preparing dry microcapsules comprising probiotic microorganisms and a carbohydrate matrix, the method comprising:
(a) providing a suspension of probiotic microorganisms;
(b) providing a matrix comprising at least one dextrin and optionally at least one disaccharide or oligosaccharide sugar; and
(c) encapsulating the suspension of probiotic microorganisms with the matrix, thereby obtaining dry microcapsules.

According to one embodiment, the process further comprises coating the microcapsules obtained from (c) with a coating composition. According to yet another embodiment, the coating composition comprises a food-grade material selected from the group consisting of: wax, shellac, resistant starch and zein.

According to yet another embodiment, a composition of the present invention further comprises a porous carrier. According to yet another embodiment, the porous carrier is selected from the group consisting of microcrystalline cellulose, spray-dried starch, and spray-dried resistant starch.

According to yet another embodiment, encapsulation comprises the step of fluidized bed air/N₂ suspension. In another embodiment, the encapsulation comprises the step of ultrasonic vacuum spray drying. In another embodiment, the encapsulation comprises the step of spray freeze-drying (also referred to as “spray freezing—freeze-drying”). In another embodiment, the encapsulation comprises a method selected from fluidized bed air/N₂ suspension, ultrasonic vacuum spray drying, and spray freeze-drying. Each possibility represents a separate embodiment of the present invention.

These and other embodiments and aspects of the present invention will become apparent in conjunction with the figures, description and claims that follow.

### BRIEF DESCRIPTION OF THE FIGURES

FIG. 1 is a schematic representation of an Ultrasonic Vacuum Spray Dryer containing three main technical components: (1) Liquid handling and spraying system; (2) Vacuum drying chamber that contains 3 heat-controlled zones (T1-T2) and a special vacuum system; and (3) Powder collection site.

FIG. 2 depicts the viability of probiotic microorganisms within microcapsules immediately after production and during the storage of the final product, up to a period of over 40 days. The following maltodextrin/trehalose formulations were used: A. Maltodextrin DE5; B. Maltodextrin DE19; C. Maltodextrin DE5: Trehalose (1:1); and D. Maltodextrin DE19: Trehalose (1:1). The encapsulated probiotics were stored at three different temperatures (4° C., 25° C. and 37° C.) and in different environments (air and N₂).

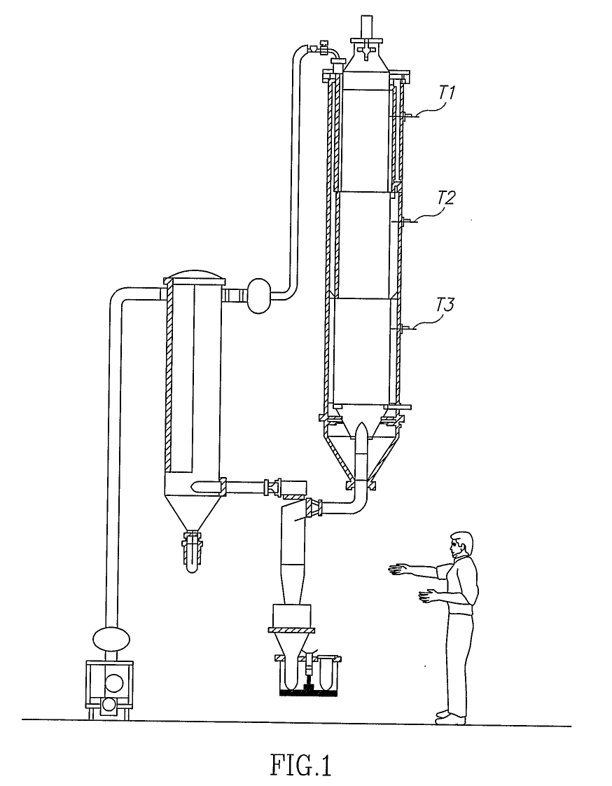

### DETAILED DESCRIPTION OF THE INVENTION

The present invention provides glassy matrices and encapsulated compositions comprising probiotic microorganisms, a dextrin, and optionally a disaccharide or oligosaccharide; microcapsules comprising same, dosage forms comprising same, and methods of manufacturing same.

In one embodiment, the present invention provides a microcapsule comprising (a) a core, the core comprising (i) a probiotic microorganism; and (ii) a dextrin, the core further being in the form of a glassy matrix, and (b) a moisture-resistant coating. In another embodiment, the core is in the form of a carbohydrate matrix. In another embodiment, the probiotic microorganism is a Lactobacillus. In another embodiment, the probiotic microorganism is a Bifidobacterium. In another embodiment, the probiotic microorganism is any other probiotic bacterium known in the art. In another embodiment, the probiotic microorganism is any probiotic yeast known in the art. Each possibility represents a separate embodiment of the present invention.

In another embodiment, the present invention provides a microcapsule comprising (a) a core, the core comprising (i) a probiotic microorganism; and (ii) a dextrin, the core further being in the form of a glassy matrix, and (b) an enteric coating. In another embodiment, the core is in the form of a carbohydrate matrix. In another embodiment, the probiotic microorganism is a Lactobacillus. In another embodiment, the probiotic microorganism is a Bifidobacterium. In another embodiment, the probiotic microorganism is any other probiotic bacterium known in the art. In another embodiment, the probiotic microorganism is any probiotic yeast known in the art. Each possibility represents a separate embodiment of the present invention.

In another embodiment, the microcapsule of methods and compositions of the present invention is manufactured by fluidized bed air/N₂ suspension. In another embodiment, the microcapsule is manufactured by ultrasonic vacuum spray drying. In another embodiment, the microcapsule is manufactured by spray freeze drying and fluidized bed air/N₂ suspension. In another embodiment, the microcapsule is manufactured by a combination of ultrasonic vacuum spray drying and fluidized bed air/N₂ suspension. In another embodiment, the microcapsule is manufactured by a combination of spray freeze drying. In another embodiment, the microcapsule is manufactured by another method known in the art that is capable of producing a glassy matrix containing probiotic bacteria and a dextrin. Each possibility represents a separate embodiment of the present invention.

In another embodiment, the core of a microcapsule of methods and compositions of the present invention further comprises a disaccharide. In another embodiment, the disaccharide is a cytoprotective disaccharide. In another embodiment, the disaccharide is trehalose. In another embodiment, the disaccharide is any other cytoprotective disaccharide known in the art. Each possibility represents a separate embodiment of the present invention.

In another embodiment, the core further comprises an oligosaccharide. In another embodiment, the oligosaccharide is a cytoprotective oligosaccharide. In another embodiment, the oligosaccharide is any other cytoprotective oligosaccharide known in the art. In another embodiment, the cytoprotective oligosaccharide is a fructo-oligo-saccharide. In another embodiment, the cytoprotective oligosaccharide is a starch. Each possibility represents a separate embodiment of the present invention.

“Cytoprotective disaccharide” and “cytoprotective oligosaccharide” refer, in another embodiment, to a disaccharide exhibiting cryopreservation activity for probiotic bacteria. In another embodiment, the terms refer to a disaccharide or oligosaccharide capable of reducing mortality of probiotic bacteria during lyophilization. In another embodiment, the terms refer to a disaccharide or oligosaccharide capable of reducing mortality of probiotic bacteria during dry storage. Each possibility represents a separate embodiment of the present invention.

The weight ratio between the dextrin and the (disaccharide or oligosaccharide) in the core or glassy matrix of methods and compositions of the present invention is, in another embodiment, within the range of 0.5:1-3:1. In another embodiment, the ratio of the dextrin to the di- or oligosaccharide is 0.3:1-3:1. In another embodiment, the ratio is 0.4:1-3:1. In another embodiment, the ratio is 0.6:1-3:1. In another embodiment, the ratio is 0.8:1-3:1. In another embodiment, the ratio is 1:1-3:1. In another embodiment, the ratio is 1.5:1-3:1. In another embodiment, the ratio is 0.5:1-2:1. In another embodiment, the ratio is 0.5:1-3.5:1. In another embodiment, the ratio is 0.5:1-2.5:1. In another embodiment, the ratio is 0.5:1-4:1. In another embodiment, the ratio is 0.5:1-5:1. In another embodiment, the ratio is 1:1. In another embodiment, the ratio is 3:2. In another embodiment, the ratio is 2:1. In another embodiment, the ratio is selected from the group consisting of: 1:1, 3:2 and 2:1. Each possibility represents a separate embodiment of the present invention.

In another embodiment, the weight ratio of total dextrin to total disaccharide+oligosaccharide in the core or glassy matrix of methods and compositions of the present invention is within the range of 0.5:1-3:1. In another embodiment, the weight ratio is 0.3:1-3:1. In another embodiment, the ratio is 0.4:1-3:1. In another embodiment, the ratio is 0.6:1-3:1. In another embodiment, the ratio is 0.8:1-3:1. In another embodiment, the ratio is 1:1-3:1. In another embodiment, the ratio is 1.5:1-3:1. In another embodiment, the ratio is 0.5:1-2:1. In another embodiment, the ratio is 0.5:1-3.5:1. In another embodiment, the ratio is 0.5:1-2.5:1. In another embodiment, the ratio is 0.5:1-4:1. In another embodiment, the ratio is 0.5:1-5:1. Each possibility represents a separate embodiment of the present invention.

The total mass of the dextrin, the disaccharide or oligosaccharide, and the microorganism is, in another embodiment, within the range of 10-40% of the total mass of the core or glassy matrix of methods and compositions of the present invention. In another embodiment, the total mass of the dextrin, the disaccharide, and the microorganism is with 10-37% of total mass of the core or glassy matrix. In another embodiment, the percentage is 10-35%. In another embodiment, the percentage is 10-32%. In another embodiment, the percentage is 10-30%. In another embodiment, the percentage is 10-27%. In another embodiment, the percentage is 10-25%. In another embodiment, the percentage is 12-22%. In another embodiment, the percentage is 10-20%. In another embodiment, the percentage is 12-40%. In another embodiment, the percentage is 15-40%. In another embodiment, the percentage is 18-40%. In another embodiment, the percentage is 20-40%. In another embodiment, the percentage is 22-40%. In another embodiment, the percentage is 25-40%. In another embodiment, the percentage is 12-38%. In another embodiment, the percentage is 15-35%. In another embodiment, the percentage is 18-32%. In another embodiment, the percentage is 20-30%. Each possibility represents a separate embodiment of the present invention.

In another embodiment, the present invention provides a glassy matrix comprising a maltodextrin, a cytoprotective disaccharide, and a probiotic microorganism.

### Probiotic Organisms

In another embodiment, the probiotic organism of methods and compositions of the present invention is a bacteria strain. In another embodiment, the probiotic organism is a yeast strain. In another embodiment, the probiotic organism is a Lactobacillus strain. In another embodiment, the probiotic organism is Lactobacillus paracasei. In another embodiment, the probiotic organism is Lactobacillus acidophilus. In another embodiment, the probiotic organism is any other Lactobacillus strain known in the art. In another embodiment, the probiotic organism is a Bifidobacterium strain. Each possibility represents a separate embodiment of the present invention.

“Probiotic” is used herein to refer to an organism with potential health benefit to a subject. The term “probiotic microorganisms” and “probiotics” are interchangeably used herein, in another embodiment, to describe probiotic bacteria and probiotic yeast. In another embodiment, the probiotic microorganism is a bacterium. In another embodiment, the probiotic microorganism is a Bifidobacterium. In another embodiment, the probiotic microorganism is Lactobacillus. In another embodiment, the probiotic microorganism is Bifidobacterium infantis. In another embodiment, the probiotic microorganism is Lactobacillus plantarum. In another embodiment, the probiotic microorganism is Bifidobacterium animalis. In another embodiment, the probiotic microorganism is Bifidobacterium animalis subsp animalis (B. animalis). In another embodiment, the probiotic microorganism is Bifidobacterium animalis subsp lactis (B. lactis). In another embodiment, the probiotic microorganism is Bifidobacterium bifidum. In another embodiment, the probiotic microorganism is Bifidobacterium breve. In another embodiment, the probiotic microorganism is Bifidobacterium infantis. In another embodiment, the probiotic microorganism is Bifidobacterium longum. In another embodiment, the probiotic microorganism is Lactobacillus acidophilus. In another embodiment, the probiotic microorganism is Lactobacillus casei. In another embodiment, the probiotic microorganism is Lactobacillus plantarum. In another embodiment, the probiotic microorganism is Lactobacillus reuteri. In another embodiment, the probiotic microorganism is Lactobacillus rhamnosus. In another embodiment, the probiotic microorganism is Lactobacillus GG.

In another embodiment, the probiotic microorganism is a yeast. In another embodiment, the probiotic microorganism is Saccharomyces boulardii.

In another embodiment, the probiotic microorganism is a lactic acid bacterium. “Lactic acid bacteria” refers, in another embodiment, to a Glade of Gram positive, low-GC, acid tolerant, non-sporulating, non-respiring rod or cocci that are associated by their common metabolic and physiological characteristics. These bacteria, usually found in decomposing plants and lactic products produce lactic acid as the major metabolic endproduct of carbohydrate fermentation. In another embodiment, the lactic acid bacterium is selected from the genera Lactobacillus, Leuconostoc, Pediococcus, Lactococcus, and Streptococcus.

Each probiotic organism represents a separate embodiment of the present invention.

In another embodiment, the glassy matrix of methods and compositions of the present invention is manufactured by fluidized bed air suspension or fluidized bed N₂ suspension (herein referred to collectively as “fluidized bed air/N₂ suspension). In another embodiment, the glassy matrix is manufactured by ultrasonic vacuum spray drying. In another embodiment, the glassy matrix is manufactured by spray freeze drying. In another embodiment, the glassy matrix is manufactured by another method known in the art that is capable of producing a glassy matrix containing probiotic bacteria and a dextrin. Each possibility represents a separate embodiment of the present invention.

### Dextrins

The dextrin of methods and compositions of the present invention is, in another embodiment, a maltodextrin. Maltodextrin exhibits a higher glass transition temperature, a decreased tendency to hydrogen bond with cell membranes, and increased potency for penetration of cell membranes, compared to the lower molecular weight sugars relative to sugars with lower molecular weight. In another embodiment, cyclodextrin is utilized. In another embodiment, a starch is utilized. In another embodiment, the dextrin of methods and compositions of the present invention exhibits a glass transition temperature higher than room temperature. In another embodiment, the dextrin of methods and compositions of the present invention exhibits a glass transition temperature comparable to that of maltodextrin. In another embodiment, the dextrin of methods and compositions of the present invention exhibits a tendency to hydrogen bond with cell membranes comparable to that of maltodextrin. In another embodiment, the dextrin of methods and compositions of the present invention exhibits an ability to penetrate cell membranes comparable to that of maltodextrin. Each possibility represents a separate embodiment of the present invention.

In another embodiment, the glass transition temperature of the dextrin utilized in methods and compositions of the present invention is between about 60-140° C. In another embodiment, the glass transition temperature is about 50-140° C. In another embodiment, the temperature is about 50-140° C., In another embodiment, the temperature is about 60-150° C. In another embodiment, the temperature is about 70-140° C. In another embodiment, the temperature is about 80-140° C. In another embodiment, the temperature is about 60-130° C. In another embodiment, the temperature is about 60-120° C. In another embodiment, the temperature is about 60-110° C. In another embodiment, the glass transition temperature is about 60-100° C.

In another embodiment, a solution or suspension utilized in methods and compositions of the present invention has a glass transition temperature between about 60-140° C. In another embodiment, the glass transition temperature is about 50-140° C. In another embodiment, the temperature is about 50-140° C. In another embodiment, the temperature is about 60-150° C. In another embodiment, the temperature is about 70-140° C. In another embodiment, the temperature is about 80-140° C. In another embodiment, the temperature is about 60-130° C. In another embodiment, the temperature is about 60-120° C. In another embodiment, the temperature is about 60-110° C. In another embodiment, the glass transition temperature is about 60-100° C.

Methods for determining the glass transition temperature of a sugar and its ability to hydrogen bond with a cell membrane are well known in the art, and are described, for example, in Taylor LS, Zografi G. J Pharm Sci. 1998 December; 87(12):1615-21). Each method represents a separate embodiment of the present invention.

### Dextrose Equivalent

The dextrose equivalent (DE) of the maltodextrin or other dextrin of methods and compositions of the present invention is, in another embodiment, within the range of 2-20. In another embodiment, the DE is from 3-20. In another embodiment, the DE is from 4-20. In another embodiment, the DE is from 5-20. In another embodiment, the DE is from 6-20. In another embodiment, the DE is from 7-20. In another embodiment, the DE is from 8-20. In another embodiment, the DE is from 10-20. In another embodiment, the DE is from 2-25. In another embodiment, the DE is from 3-25. In another embodiment, the DE is from 4-25. In another embodiment, the DE is from 5-25. In another embodiment, the DE is from 6-25. In another embodiment, the DE is from 7-25. In another embodiment, the DE is from 8-25. In another embodiment, the DE is from 10-25. In another embodiment, the DE is from 12-25. In another embodiment, the DE is from 2-30. In another embodiment, the DE is from 3-30. In another embodiment, the DE is from 4-30. In another embodiment, the DE is from 5-30. In another embodiment, the DE is from 6-30. In another embodiment, the DE is from 7-30. In another embodiment, the DE is from 8-30. In another embodiment, the DE is from 10-30. In another embodiment, the DE is from 12-30. In another embodiment, the DE is from 2-18. In another embodiment, the DE is from 2-16. In another embodiment, the DE is from 2-15. In another embodiment, the DE is from 2-14. In another embodiment, the DE is from 2-12. In another embodiment, the DE is from 2-10. In another embodiment, the DE is from 3-18. In another embodiment, the DE is from 4-16. In another embodiment, the DE is from 5-15. In another embodiment, the DE is from 6-14. In another embodiment, the DE is from 8-12. Each possibility represents a separate embodiment of the present invention.

“Dextrose equivalent” is defined as the number of glycosidic bonds cleaved divided by the total number of glycosidic bonds present, using the following conditions.

In another embodiment, DE is defined as:

$$DE = 100 \times ( \frac{Number of glycosidic bonds cleaved}{Initial number of glycosidic bonds present} )$$

In another embodiment, DE is determined analytically by use of the closely related, but not identical, expression:

DE=100×(reducing sugar, expressed as glucose)/total carbohydrate

In another embodiment, DE represents the percentage hydrolysis of the glycosidic linkages present. Pure glucose has a DE of 100, pure maltose has a DE of about 50 (depending upon the analytical methods used) and starch has a DE of effectively zero.

Methods for determining the dextrose equivalent of a maltodextrin are well known in the art, and are described, for example, in Wangsakan A, Chinachoti P, McClements DJ. J Agric Food Chem. 2003 Dec. 17; 51(26):7810-4; and Chronakis IS. Crit Rev Food Sci Nutr. 1998 October; 38(7):599-637.

In another embodiment, dextrose equivalent is determined using a titration apparatus and Fehling's Solution, as described, for example, in Lane, J. H., Eynon, L. (1923). Determination of reducing sugars by means of Fehling's solution with methylene blue as internal indicator. J. Soc. Chem. Ind. Trans. 32-36.

### Glassy Matrices and Glass Transition Temperature

“Glassy matrix” refers, in another embodiment, to a matrix that is solid at room temperature and exhibits high elastic modulus and strength. Glassy states are well known in the art, and are described, for example, in H. Levine and L. Slade, “Glass Transitions in Foods”, pgs. 83-205 in Physical Chemistry of Foods, H. Schwartzberg and R. Hartel, Eds., Marciel Dekker, New York, 1992; and H. Levine and L. Slade, “Water as a Plasticizer: physico-chemical aspects of low-moisture polymeric systems”, pgs. 79-185 in Water Science Reviews, Vol. 3, F. Franks, Ed., Cambridge University Press, London, 1988. In another embodiment, the term refers to a solid matrix having a rigid configuration and lacking a regular atomic arrangement. In another embodiment, the term refers to a solid matrix wherein molecular chains or coils are effectively frozen, but not in a regular pattern. “Glassy matrices” of the present invention need not include either silicon dioxide or arsenic. In another embodiment, the glassy matrix of methods and compositions of the present invention is a carbohydrate-based glassy matrix.

In another embodiment, a glassy matrix of methods and compositions of the present invention exhibits sufficiently high glass transition temperature (T_{g}) such that the glassy matrix is stable at ambient temperatures. The relationship between the glass transition temperature and moisture content for a matrix is described by Y. Roos and M. Karel, J. Food Science, Vol. 56(6), 1676-1681 (1991). T_{g}, the glass transition temperature, increases with decreasing moisture content or increasing molecular weight of the maltodextrin. In another embodiment, the Tg is greater than 30° C. In another embodiment, the Tg is greater than 35° C. In another embodiment, the Tg is greater than 40° C. In another embodiment, the Tg is greater than 50° C. In another embodiment, the Tg is greater than 60° C.

“Glass transition temperature” or “T_{g}” refers, in another embodiment, to the temperature below which the physical properties of amorphous materials vary in a manner similar to those of a solid phase (glassy state), and above which amorphous materials behave like liquids (rubbery state). In another embodiment, the term refers to, the temperature below which molecules have little relative mobility.

Each definition of “glassy matrix” and “T_{g}” represents a separate embodiment of the present invention.

Methods of measuring glassiness are well known in the art, and include, for example, differential scanning calorimetry (DSC), as described, for example, in Chang Z H, Baust J G. Cryobiology. 1991 February; 28(1):87-95). Each method represents a separate embodiment of the present invention.

Without wishing to be bound by any particular theory or mechanism of action, it is possible to conceive that the glassy environment that encapsulates the microorganisms may be responsible for the exceptional stability, both at high temperatures during drying processes, e.g. spray drying and extrusion fixation at high temperatures in high velocity gas streams, as well as storage of the composition

### Moisture-Resistant Coatings

“Moisture-resistant” refers, in another embodiment, to an ability of a coating to protect the core containing therein from external moisture. In another embodiment, moisture resistance is measured by the leakage of water-soluble materials from the capsule in aqueous environment. In another embodiment, moisture resistance is measured by a moisture sorption assay. Methods for measuring moisture resistance are well known in the art, and are described, for example, in Pereira de Souza T et al (Eudragit E as excipient for production of granules and tablets from Phyllanthus niruri L spray-dried extract. AAPS PharmSciTech. 2007 Apr. 27; 8(2):Article 34); Young P M et al, Interaction of moisture with sodium starch glycolate. Pharm Dev Technol. 2007; 12(2):211-6); and de la Luz Reus Medina M et al, Comparative evaluation of powder and tableting properties of low and high degree of polymerization cellulose I and cellulose II excipients. Int J. Pharm. 2007 Jun. 7; 337(1-2):202-9). Each method represents a separate embodiment of the present invention.

The moisture-resistant coating of methods and compositions of the present invention is, in another embodiment, a waxy coating. In another embodiment, the coating of methods and compositions of the present invention is resistant to oxygen penetration. Each possibility represents a separate embodiment of the present invention.

### Waxes

“Wax” and “waxy,” as used herein refer, in one embodiment, to an ester of a long-chain carboxylic acid, typically C₁₆ or greater, with a long-chain alcohol. In another embodiment, a wax of methods and compositions of the present invention is a substance that is solid at room temperature and has a melting point under about 100° C. In another embodiment, the wax exhibits a “waxy” feel. In another embodiment, the wax is moisture-resistant.

In another embodiment, the wax is polyethylene glycol. In another embodiment, the wax is paraffin. In another embodiment, the wax is palm oil. In another embodiment, the wax is hydrogenated cottonseed oil. In another embodiment, the wax is carnauba wax. In another embodiment, the wax is hydrogenated castor oil. In another embodiment, the wax is a mono glyceride. In another embodiment, the wax is a di glyceride. In another embodiment, the wax is a combination of a mono glyceride and a di glyceride. In another embodiment, the wax is an animal wax. In another embodiment, the wax is an insect wax. In another embodiment, the wax is a hydrogenated vegetable oil. In another embodiment, the wax is a plant wax. In another embodiment, the wax is a mineral wax. In another embodiment, the wax is a petroleum wax. In another embodiment, the wax is a synthetic wax. Each possibility represents a separate embodiment of the present invention.

In another embodiment, the wax of methods and compositions of the present invention is a polyethylene-based wax. In another embodiment, the wax is a Fischer-Tropsch wax. In another embodiment, the wax is a chemically modified wax. In another embodiment, the wax is an esterified wax. In another embodiment, the wax is a substituted amide wax. In another embodiment, the wax is a polymerized α-olefin.

In another embodiment, the wax is paraffin wax. In another embodiment, the wax is a microcrystalline wax. In another embodiment, the wax is an anionic emulsifying wax. In another embodiment, the wax is an ionic emulsifying wax. In another embodiment, the wax is a bleached wax. In another embodiment, the wax is carnauba wax. In another embodiment, the wax is a cetyl ester. In another embodiment, the wax is a hard wax. In another embodiment, the wax is a refined wax. In another embodiment, the wax is a white wax. In another embodiment, the wax is a yellow wax.

### Dry Cores/Matrices

In another embodiment, the core or glassy matrix of methods and compositions of the present invention is a dry core or dry glassy matrix. The terms “dry” and “dry food product,” as used herein, refers, in another embodiment, to a water activity at room temperature below 0.4. In another embodiment, the core or glassy matrix has a water activity at room temperature of below 0.25. In another embodiment, the water activity is below 0.35. In another embodiment, the water activity is below 0.35. In another embodiment, the water activity is below 0.30. In another embodiment, the water activity is below 0.2. In another embodiment, the water activity is below 0.18. In another embodiment, the water activity is below 0.15. In another embodiment, the water activity is 0.4 or less. In another embodiment, the water activity is 0.35 or less. In another embodiment, the water activity is 0.3 or less. In another embodiment, the water activity is 0.25 or less. In another embodiment, the water activity is 0.2 or less. In another embodiment, the water activity is 1.8 or less. In another embodiment, the water activity is below the water activity threshold at which the core or glassy matrix is able to retain its glassiness. Each possibility represents a separate embodiment of the present invention.

“Water activity” or a_{w}, is the energy state of water in a substance. It is defined as the vapor pressure of water divided by that of pure water at the same temperature; therefore, pure distilled water has a water activity of exactly one.

In another embodiment, “dry” refers to a level of residual moisture at or below the accepted standard for a freeze-dried product. Methods for measuring residual moisture are well known in the art, and include, for example, (1) the gravimetric or loss on drying test for residual moisture (Code of Federal Regulations, 21 CFR 610.13 (a), p. 52. U.S. Government Printing Office: Washington, D.C., 1988; May, Wheeler and Grim, Cryobiology, 26,277-284, 1989) for freeze-dried biological products, which measures the maximum loss in weight of a weighed sample equilibrated to constant weight over anhydrous phosphorus pentoxide at a pressure of not more than 1 mm of mercury and a temperature of 20° C. to 30° C. for as long as it has been established is sufficient to result in a constant weight; (2) the Karl Fischer method (May, et al., Journal of Biological Standardization, 10, 249-259, 1982); (3) Thermogravimetry (TG) and Thermogravimetry/Mass Spectrometry has also been applied to the determination of residual moisture in freeze-dried biological products (May, et. al., Journal of Biological Standardization, 10, 249-259, 1982); and (4) the Moisture Evolution Analyzer (Jewell, Workman and Zeleznick, Developments in Biological Standardization, 36, pp. 181-189, S. Karger: Basel, 1977). Each possibility represents a separate embodiment of the present invention.

The terms “dry” and “solid” are used interchangeably herein to describe a composition in a dry solid form. In another embodiment, the terms refer to a composition, core, or glassy matrix with a water activity of less than 0.4. In another embodiment, the water activity is less than 0.25. In another embodiment, the water activity is less than 0.45. In another embodiment, the water activity is less than 0.42. In another embodiment, the water activity is less than 0.38. In another embodiment, the water activity is less than 0.36. In another embodiment, the water activity is less than 0.34. In another embodiment, the water activity is less than 0.32. In another embodiment, the water activity is less than 0.3. In another embodiment, the water activity is less than 0.29. In another embodiment, the water activity is less than 0.28. In another embodiment, the water activity is less than 0.27. In another embodiment, the water activity is less than 0.26. In another embodiment, the water activity is less than 0.24. In another embodiment, the water activity is less than 0.23. In another embodiment, the water activity is less than 0.22. In another embodiment, the water activity is less than 0.21. In another embodiment, the water activity is less than 0.2. In another embodiment, the water activity is less than 0.15. In another embodiment, the water activity is less than 0.1. Each possibility represents a separate embodiment of the present invention.

In another embodiment, the water activity is within the range of 0.01-0.25. In another embodiment, the water activity is within the range of 0.02-0.25. In another embodiment, the water activity is within the range of 0.03-0.25. In another embodiment, the water activity is within the range of 0.04-0.25. In another embodiment, the water activity is within the range of 0.05-0.25. In another embodiment, the water activity is within the range of 0.06-0.25. In another embodiment, the water activity is within the range of 0.08-0.25. In another embodiment, the water activity is within the range of 0.1-0.25. In another embodiment, the water activity is within the range of 0.01-0.4. In another embodiment, the water activity is within the range of 0.02-0.4. In another embodiment, the water activity is within the range of 0.03-0.4. In another embodiment, the water activity is within the range of 0.04-0.4. In another embodiment, the water activity is within the range of 0.05-0.4. In another embodiment, the water activity is within the range of 0.06-0.4. In another embodiment, the water activity is within the range of 0.08-0.4. In another embodiment, the water activity is within the range of 0.1-0.4. Each possibility represents a separate embodiment of the present invention.

### Additional Core Components

In another embodiment, the core of methods and compositions of the present invention further comprises microcrystalline cellulose. In another embodiment, a suspension comprising the probiotic bacteria and dextrin is absorbed onto the microcrystalline cellulose; e.g. using fluidized bed air/N₂ suspension. In another embodiment, the mass of the microcrystalline cellulose is 60-90% of the total mass of the core. In another embodiment, the mass of the microcrystalline cellulose is 60-85% of the total core mass. In another embodiment, the mass of the microcrystalline cellulose is 60-80% of the total core mass. In another embodiment, the mass of the microcrystalline cellulose is 65-90% of the total core mass. In another embodiment, the mass of the microcrystalline cellulose is 70-90% of the total core mass. In another embodiment, the mass of the microcrystalline cellulose is 65-85% of the total core mass. Each possibility represents a separate embodiment of the present invention.

In another embodiment, the core of methods and compositions of the present invention further comprises a starch. In another embodiment, a starch is used in place of the microcrystalline cellulose (e.g. the suspension comprising the probiotic bacteria and dextrin is absorbed onto the starch). In another embodiment, the starch is a spray-dried starch. In another embodiment, the starch is a spray-dried resistant starch. In another embodiment, the starch is any other porous starch known in the art.

In another embodiment, tricalcium phosphate (TCP) is used in place of the microcrystalline cellulose. In another embodiment, SiO₂ (e.g. Sipernat®) is used in place of the microcrystalline cellulose. In another embodiment, calcium carbonate is used in place of the microcrystalline cellulose. Each possibility represents a separate embodiment of the present invention.

In another embodiment, the mass of the starch is 60-90% of the total mass of the core. In another embodiment, the mass of the starch is 60-85% of the total core mass. In another embodiment, the mass of the starch is 60-80% of the total core mass. In another embodiment, the mass of the starch is 65-90% of the total core mass. In another embodiment, the mass of the starch is 70-90% of the total core mass. In another embodiment, the mass of the starch is 65-85% of the total core mass. In another embodiment, a suspension comprising the probiotic bacteria and dextrin is absorbed onto the starch; e.g. using fluidized bed air/N₂ suspension. Each possibility represents a separate embodiment of the present invention.

In another embodiment, the mass of the microcrystalline cellulose, spray-dried starch, or spray-dried resistant starch is within the range of 60-90% of the total mass of the glassy matrix or core of methods and compositions of the present invention. In another embodiment, the mass of the microcrystalline cellulose or starch is from 62-90% of the total mass of the glassy matrix or core. In another embodiment, the percentage is 65-90%. In another embodiment, the percentage is 68-90%. In another embodiment, the percentage is 70-90%. In another embodiment, the percentage is 72-90%. In another embodiment, the percentage is 75-90%. In another embodiment, the percentage is 60-88%. In another embodiment, the percentage is 60-85%. In another embodiment, the percentage is 60-82%. In another embodiment, the percentage is 60-80%. In another embodiment, the percentage is 60-78%. In another embodiment, the percentage is 60-75%. In another embodiment, the percentage is 62-88%. In another embodiment, the percentage is 65-85%. In another embodiment, the percentage is 68-82%. In another embodiment, the percentage is 70-80%. Each possibility represents a separate embodiment of the present invention.

### Additional Coatings

The core, glassy matrix, or microcapsule of methods and compositions of the present invention is, in another embodiment, coated with a food-grade coating. In another embodiment, the food-grade coating is a moisture-resistant coating. In another embodiment, the food-grade coating is an oxidation-resistant coating. In another embodiment, the food-grade coating is an enteric coating. In another embodiment, the food-grade coating is any other type of food-grade coating known in the art. In other embodiments, such coatings ensure the release of the microorganisms only after their arrival at the intestine, while protecting the microorganisms from the environment in the stomach. In other embodiments, the coating improves the survival prospects of bacterial cells in the gastrointestinal (GI) tract. Each possibility represents a separate embodiment of the present invention.

In another embodiment, a method of the present invention further comprises the step of coating the microcapsules with a food-grade coating. In another embodiment, the food-grade coating is a food-grade enteric coating. In another embodiment, the food-grade coating is a moisture-resistant coating. In another embodiment, the food-grade coating is an oxidation-resistant coating. Each possibility represents a separate embodiment of the present invention.

The food-grade coating of methods and compositions of the present invention comprises, in another embodiment, wax, e.g. as defined hereinabove. In another embodiment, the food-grade coating comprises shellac. In another embodiment, the food-grade coating comprises resistant starch. In another embodiment, the food-grade coating comprises zein protein. In another embodiment, the food-grade coating comprises ethylcellulose. In another embodiment, the food-grade coating comprises methylcellulose. In another embodiment, the food-grade coating comprises hydroxypropyl methylcellulose. In another embodiment, the food-grade coating comprises amylose acetate phthalate. In another embodiment, the food-grade coating comprises cellulose acetate phthalate. In another embodiment, the food-grade coating comprises hydroxyl propyl methyl cellulose phthalate. In another embodiment, the food-grade coating comprises an ethylacrylate. In another embodiment, the food-grade coating comprises a methylmethacrylate. In another embodiment, the food-grade coating consists of one of the above compounds. Each possibility represents a separate embodiment of the present invention.

In another embodiment, the food grade coating comprises a material selected from the group consisting of: wax, shellac, resistant starch, zein protein, ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate. Each possibility represents a separate embodiment of the present invention.

In other embodiments, RS suspensions in distilled water, Zein protein in ethanol, or Ethylcellulose (ETHOCEL®) in acetone are used as food-grade enteric coatings for delivery of bioactive materials such as probiotics to the GI tract or specifically to the colon.

In another embodiment, wax is used as one of the coating materials in “multi-layer” walls in order to improve shelf life of the final product as well as pre-coater before Ethylcellulose (ETHOCEL®).

In another embodiment, RS, zein protein, wax, and/or Ethylcellulose (ETHOCEL®) are sprayed through a nozzle onto the particles to be coated and film formation is initiated. This is followed by a succession of drying and wetting stages.

### Compositions and Dosage Forms

In another embodiment, the present invention provides a composition comprising a microcapsule or glassy matrix of the present invention. In another embodiment, the present invention provides a foodstuff comprising a microcapsule or glassy matrix of the present invention. In another embodiment, the present invention provides a dry food mix comprising a microcapsule or glassy matrix of the present invention. Each possibility represents a separate embodiment of the present invention.

In another embodiment, the present invention provides a dosage form wherein a probiotic-containing core or glassy matrix is coated with a wax-containing coat. In another embodiment, the dosage form comprises, in addition to the wax-containing coat, an enteric coat. In another embodiment, the enteric coat is an ethylcellulose-containing coat. In another embodiment, the enteric coat is another food-grade coat disclosed herein.

The compositions of the invention are particularly useful for applications in the food industry. In other embodiments, probiotic microorganisms in dry compositions of the invention are added as coated or uncoated microcapsules to dry food products.

### Methods of Preparing Microcapsules

In another embodiment, the present invention provides a method of preparing microcapsules, comprising the steps of: (a) applying a suspension, the solution comprising a solvent, a probiotic microorganism, and a solubilized dextrin, wherein the dextrin is capable of forming a glassy matrix, to fluidized particles, the fluidized particles comprising a porous polymer carrier, thereby generating wetted particles; (b) initiating film formation by simultaneously subjecting the wetted particles to a drying process, thereby generating coated particles; and (c) optionally applying additional layers of the solution to the coated particles, thereby preparing microcapsules. In another embodiment, the matrix in the microcapsules is a carbohydrate matrix. In another embodiment, the suspension further comprises a cytoprotective disaccharide, as defined herein. In another embodiment, the disaccharide is trehalose. In another embodiment, the dextrin is a maltodextrin. Each possibility represents a separate embodiment of the present invention.

In another embodiment, the suspension further comprises a disaccharide, as defined herein. In another embodiment, the suspension further comprises an oligosaccharide, as defined herein. In another embodiment, the suspension further comprises a solubilized disaccharide or oligosaccharide.

In another embodiment, the weight ratio between the probiotic microorganism and the soluble components of the suspension is one of the values or within one of the ranges defined above for cores and glassy matrices of the present invention.

### Porous Polymer Carriers

The porous polymer carrier of methods and compositions of the present invention is, in another embodiment, a cellulose. In another embodiment, the cellulose is a microcrystalline cellulose. In another embodiment, the cellulose is any other porous cellulose known in the art. In another embodiment, the porous polymer carrier is a starch. In another embodiment, the starch is a spray-dried starch. In another embodiment, the starch is a resistant starch. In another embodiment, the starch is a spray-dried resistant starch. In another embodiment, the starch is any other porous starch known in the art. Each possibility represents a separate embodiment of the present invention.

“Resistant starch” refers, in another embodiment, to starch that escapes digestion in the small intestine of healthy individuals. Some carbohydrates, such as sugars and most starch, are rapidly digested and absorbed as glucose into the body through the small intestine and subsequently used for short-term energy needs or stored. Resistant starch, on the other hand, resists digestion and passes through to the large intestine where it acts like dietary fiber.

In another embodiment, the resistant starch of methods and compositions of the present invention is RS2, defined as resistant starch that occurs in its natural granular form, such as uncooked potato, green banana flour and high amylose corn. In another embodiment, the resistant starch is RS3, defined as resistant starch that is formed when starch-containing foods are cooked and cooled such as in bread, cornflakes and cooked-and-chilled potatoes or retrograded high amylose corn. In another embodiment, the resistant starch is RS4, defined as starches that have been chemically modified to resist digestion. This type of resistant starches can have a wide variety of structures and are not found in nature.

The average particle size of the food-grade porous powder utilized in methods and compositions of the present invention is, in another embodiment, at least 20 micrometer (mcm). In another embodiment, the average particle size is at least 10 mcm. In another embodiment, the average particle size is at least 12 mcm. In another embodiment, the average particle size is at least 15 mcm. In another embodiment, the average particle size is at least 25 mcm. In another embodiment, the average particle size is at least 30 mcm. Each possibility represents a separate embodiment of the present invention.

### Drying Processes

The drying process of methods and compositions of the present invention comprises, in another embodiment, the step of contacting the wetted particles with warm air. In another embodiment, the drying process is a fluidized bed air process. In another embodiment, the drying process comprises a fluidized bed air process. Each possibility represents a separate embodiment of the present invention.

In another embodiment, the drying process comprises the steps of (a) spraying a suspension or composition comprising the wetted particles into a vacuum chamber; and (b) evaporating the remaining solvent from the wetted particles in a fluidized bed. In another embodiment, the drying process is an ultrasonic vacuum spray drying process. In another embodiment, the drying process comprises an ultrasonic vacuum spray drying process. Each possibility represents a separate embodiment of the present invention.

In another embodiment, the drying process comprises the steps of (a) spraying a suspension or composition comprising the wetted particles into liquid nitrogen; and (b) freeze-drying the mixture resulting from step (a) for 1-3 days. In another embodiment, the drying process is a spray drying/freeze drying process. In another embodiment, the drying process comprises a spray drying/freeze drying process. Each possibility represents a separate embodiment of the present invention.

In accordance with the present invention, it has been unexpectedly discovered that probiotic microorganisms can be stored in dry form while retaining high viability of the stored probiotics. In some cases, at least 70% of the organisms are viable after encapsulation and over 75% of this population remains viable after about 40 days of storage. In other cases, compositions of the invention enable high viability of the probiotics during thermal processing of the product, even at temperatures exceeding those commonly used for handling viable probiotics.

In another embodiment, at least 50% of the organisms are viable after encapsulation. In another embodiment, the percentage is at least 55%. In another embodiment, the percentage is at least 60%. In another embodiment, the percentage is at least 65%. In another embodiment, the percentage is at least 75%. In another embodiment, the percentage is at least 80%. Each possibility represents a separate embodiment of the present invention.

In another embodiment, at least 50% of the number of viable organisms after encapsulation remain viable after about 40 days of storage. In another embodiment, the percentage is at least 55%. In another embodiment, the percentage is at least 60%. In another embodiment, the percentage is at least 65%. In another embodiment, the percentage is at least 75%. In another embodiment, the percentage is at least 80%. Each possibility represents a separate embodiment of the present invention.

Compositions of the invention can be prepared, in another embodiment, by any technology suitable to form microcapsules on an industrial scale, while protecting the viability of the probiotic microorganisms. Preferably, such techniques include ultrasonic spray dryer, fluidized bed coating and spray freeze-drying (SFD).

### Coating Technologies

### Fluidized Bed Air/Nitrogen Processor

This technology is used for two purposes: probiotic encapsulation and coating probiotic microorganisms entrapped in a glassy matrix.

For probiotic encapsulation, a food-grade porous polymer carrier is used, e.g. microcrystalline cellulose. In another embodiment, spray-dried starch is utilized. In another embodiment, spray-dried resistant starch is utilized. In another embodiment, another food-grade carrier is utilized. Probiotic microorganisms are adsorbed, in this method, into/onto the porous carrier. Each possibility represents a separate embodiment of the present invention.

In another embodiment, the resulting microparticles are subsequently further coated, using fluidized bed air/nitrogen technology, with RS III, ETHOCEL®, or zein protein and/or additional layers of food-grade wall materials (such as a wax layer for preventing moisture and oxygen penetration).

Methods for using coating cores with RS (resistant starch) are well known in the art and are described, for example, in Shimoni et al., Innovative Food Science and Emerging Technologies, 5:93-100, 2004). Preferably, the RS suspension is homogenized before use, in order to reduce particle size. Preparation of RS from high amylose cornstarch is described in Shimoni et al., Carbohydrate Polymers, 54(3): 363-369, 2003.

Fluid bed spray coating is, in another embodiment, a three-step process. First, the particles to be coated are fluidized in the warm atmosphere of the coating chamber. Then, the coating material is sprayed through a nozzle onto the particles and film formation is initiated, followed by a succession of drying and wetting stages. The small droplets of the sprayed liquid comprising probiotic microorganisms or the coating material spread onto the particle surface of the microcrystalline cellulose or microcapsules, and coalesce. The solvent or the mixtures are then evaporated by the warm air or nitrogen gas, and the coating material adheres to the particles.

The average size of microcapsules manufactured by using fluidized bed air/nitrogen technology is, in another embodiment, at least 20 micrometer (mcm). In another embodiment, the average particle size is 200-250 mcm. In another embodiment, the average particle size is 150-200 mcm. In another embodiment, the average particle size is 100-150 mcm. In another embodiment, the average particle size is 70-100 mcm. In another embodiment, the average particle size is 50-70 mcm. In another embodiment, the average particle size is 30-50 mcm. In another embodiment, the average particle size is 20-30 mcm. In another embodiment, the average particle size is 15-20 mcm. In another embodiment, the average particle size is 10-15 mcm. In another embodiment, the average particle size is at least 10 mcm. In another embodiment, the average particle size is at least 10 mcm. In another embodiment, the average particle size is at least 15 mcm. In another embodiment, the average particle size is at least 25 mcm. In another embodiment, the average particle size is at least 30 mcm. Each possibility represents a separate embodiment of the present invention.

### Ultrasonic Vacuum Spray Drying Process

More gentle heating is achieved by this technique, since the vacuum in the drier space significantly reduces the temperature of the product as well as the particles' residence time.

The Ultrasonic Vacuum Spray Dryer is disclosed in U.S. Pat. No. 5,624,530 and is also available from USDryer, Migdal Haemek, Israel. The technique includes an ultrasonic atomizer, which can operate in a vacuum environment, and a vacuum chamber with adjustable heating zones. The atomized spray is directed into a vacuum chamber whose internal temperature control is set according to the specific task required. The drying is performed in two stages. In the first stage, the homogeneous drops fall free in the vacuum chamber within 4-5 seconds and lose 90-95% of their free water, and the drops' temperature does not exceed 20-30° C. During the second drying stage in a cooled (10-15° C.) vacuum-Nitrogen fluidized-bed, the remaining free water and any parts of coupling water evaporate within 20-60 min. After this stage, the product is removed from the collector without stopping the process.

Subsequently, the dried particles, wherein the probiotic microorganisms are entrapped in a matrix form, are coated using “fluidized bed air/nitrogen processor” technology (e.g. by RS, zein protein, wax, and/or ETHOCEL) as described above.

The average size of microcapsules manufactured by ultrasonic vacuum spray drying technology is, in another embodiment, at least 20 micrometer (mcm). In another embodiment, the average particle size is 20-50 mcm. In another embodiment, the average particle size is 20-40 mcm. In another embodiment, the average particle size is 30-50 mcm. In another embodiment, the average particle size is 20-60 mcm. In another embodiment, the average particle size is 15-50 mcm. In another embodiment, the average particle size is 20-80 mcm. In another embodiment, the average particle size is 20-30 mcm. In another embodiment, the average particle size is 15-20 mcm. In another embodiment, the average particle size is 10-15 mcm. In another embodiment, the average particle size is at least 10 mcm. In another embodiment, the average particle size is at least 10 mcm. In another embodiment, the average particle size is at least 15 mcm. In another embodiment, the average particle size is at least 25 mcm. In another embodiment, the average particle size is at least 30 mcm. Each possibility represents a separate embodiment of the present invention.

### Spray Freezing—Freeze-Drying Process

Relatively fast freezing rates are typically achieved by this technology. A suspension of probiotic microorganisms is sprayed by a nozzle into freezing liquid nitrogen. The frozen particles are further freeze dried by conventional freeze-drying equipment for 24-48 hours.

Subsequently, the dried particles, wherein the probiotic microorganisms are entrapped in a matrix form, are coated using “fluidized bed air/nitrogen processor” technology (e.g. by RS, zein protein, wax, and/or ETHOCEL®) as described above.

The average size of microcapsules manufactured by spray freezing—freeze-drying technology is, in another embodiment, in the range of 0.5-1.7 mm. In another embodiment, the average particle size is 0.6-1.6 mm. In another embodiment, the average particle size is 0.7-1.4 mm. In another embodiment, the average particle size is 0.5-2 mm. In another embodiment, the average particle size is 0.5-2.5 mm. In another embodiment, the average particle size is 0.4-0.8 mm. In another embodiment, the average particle size is 0.3-0.6 mm. In another embodiment, the average particle size is 0.2-0.4 mm. In another embodiment, the average particle size is 0.1-0.2 mm. In another embodiment, the average particle size is 50-100 mcm. In another embodiment, the average particle size is 30-50 mcm. In another embodiment, the average particle size is at least 0.5 mm. In another embodiment, the average particle size is at least 0.4 mm. In another embodiment, the average particle size is at least 0.3 mm. In another embodiment, the average particle size is at least 0.2 mm. In another embodiment, the average particle size is at least 0.15 mm. In another embodiment, the average particle size is at least 0.1 mm. In another embodiment, the average particle size is at least 70 mcm. In another embodiment, the average particle size is at least 50 mcm. In another embodiment, the average particle size is at least 40 mcm. In another embodiment, the average particle size is 20-30 mcm. In another embodiment, the average particle size is at least 20 mcm. In another embodiment, the average particle size is at least 25 mcm. In another embodiment, the average particle size is at least 30 mcm. Each possibility represents a separate embodiment of the present invention.

### Weight Ratios

The weight ratio between the bacteria and the other dry components of the glassy matrix or core of methods and compositions of the present invention is, in another embodiment, within the range of 0.5%-30%. In another embodiment, the weight ratio is within the range 0.4-30%. In another embodiment, the weight ratio is within the range 0.6-30%. In another embodiment, the weight ratio is within the range 0.8-30%. In another embodiment, the weight ratio is within the range 1-30%. In another embodiment, the weight ratio is within the range 1.5-30%. In another embodiment, the weight ratio is within the range 2-30%. In another embodiment, the weight ratio is within the range 3-30%. In another embodiment, the weight ratio is within the range 0.5-25%. In another embodiment, the weight ratio is within the range 0.5-20%. In another embodiment, the weight ratio is within the range 0.5-15%. In another embodiment, the weight ratio is within the range 0.5-12%. In another embodiment, the weight ratio is within the range 0.5-10%. In another embodiment, the weight ratio is within the range 0.6-25%. In another embodiment, the weight ratio is within the range 0.7-20%. In another embodiment, the weight ratio is within the range 0.8-20%. In another embodiment, the weight ratio is within the range 1-20%. In another embodiment, the weight ratio is within the range 1.5-20%. Each possibility represents a separate embodiment of the present invention.

In another embodiment, for compositions and methods wherein fluidized bed air/N₂ suspension is utilized, the weight ratio between the bacteria and the other dry components of the glassy matrix or core is 5-30%. In another embodiment, the weight ratio is 4-30%. In another embodiment, the weight ratio is 6-30%. In another embodiment, the weight ratio is 8-30%. In another embodiment, the weight ratio is 10-30%. In another embodiment, the weight ratio is 5-25%. In another embodiment, the weight ratio is 5-20%. In another embodiment, the weight ratio is 5-15%. Each possibility represents a separate embodiment of the present invention.

In another embodiment, for compositions and methods wherein ultrasonic vacuum spray drying is utilized (optionally followed by used of fluidized bed air/N₂ suspension to add additional coatings), the weight ratio between the bacteria and the other dry components of the glassy matrix or core is 0.5-10%. In another embodiment, the weight ratio is 0.4-10%. In another embodiment, the weight ratio is 0.6-10%. In another embodiment, the weight ratio is 0.7-10%. In another embodiment, the weight ratio is 0.8-10%. In another embodiment, the weight ratio is 1-10%. In another embodiment, the weight ratio is 1.5-10%. In another embodiment, the weight ratio is 2-10%. In another embodiment, the weight ratio is 0.5-12%. In another embodiment, the weight ratio is 0.5-8%. In another embodiment, the weight ratio is 0.5-7%. In another embodiment, the weight ratio is 0.5-6%. In another embodiment, the weight ratio is 0.6-8%. In another embodiment, the weight ratio is 0.8-7%. In another embodiment, the weight ratio is 1-6%. Each possibility represents a separate embodiment of the present invention.

In another embodiment, for compositions and methods wherein spray freeze drying is utilized (optionally followed by used of fluidized bed air/N₂ suspension to add additional coatings), the weight ratio between the bacteria and the other dry components of the glassy matrix or core is 0.5-10%. In another embodiment, the weight ratio is 0.4-10%. In another embodiment, the weight ratio is 0.6-10%. In another embodiment, the weight ratio is 0.7-10%. In another embodiment, the weight ratio is 0.8-10%. In another embodiment, the weight ratio is 1-10%. In another embodiment, the weight ratio is 1.5-10%. In another embodiment, the weight ratio is 2-10%. In another embodiment, the weight ratio is 0.5-12%. In another embodiment, the weight ratio is 0.5-8%. In another embodiment, the weight ratio is 0.5-7%. In another embodiment, the weight ratio is 0.5-6%. In another embodiment, the weight ratio is 0.6-8%. In another embodiment, the weight ratio is 0.8-7%. In another embodiment, the weight ratio is 1-6%. Each possibility represents a separate embodiment of the present invention.

### Example 1

### Formulations

Examples of core formulations containing maltodextrins, optionally in combination with trehalose, resistant starch (RS), and/or microcrystalline cellulose (the latter only with the “fluidized bed air processor” technology), are listed in Table 1. The amount of microorganisms used depends on the number of probiotic microorganisms required to be absorbed onto the microcrystalline cellulose.

TABLE 1
Different core formulations used in the three technologies. The RS-
containing suspensions in Table 1 (i.e. the last 3 compositions) were used
for preparing the core matrix by the spray freezing - freeze
drying technology.

| Maltodextrin | Trehalose | RS | Solids conc. |
|---|---|---|---|
| [% w/v] | [% w/v] | [% w/v] | [% w/v] |
| 10 | 0 | 0 | 10 |
| 20 | 0 | 0 | 20 |
| 30 | 0 | 0 | 30 |
| 40 | 0 | 0 | 40 |
| 10 | 10 | 0 | 20 |
| 10 | 20 | 0 | 30 |
| 15 | 15 | 0 | 30 |
| 20 | 10 | 0 | 30 |
| 20 | 20 | 0 | 40 |
| 30 | 20 | 0 | 50 |
| 0 | 30 | 0 | 30 |
| 0 | 5 | 5 | 10 |
| 0 | 10 | 10 | 20 |
| 0 | 0 | 10 | 10 |

For maltodextrin/trehalose formulations, distilled water was heated to at least 93° C. prior to addition of maltodextrin and trehalose, in order to obtain complete dissolution of maltodextrin.

Resistant starch (RS) III was prepared by dissolving high amylose cornstarch in distilled water at room temperature, followed by thermal treatment (120° C. for 120 min) and incubation overnight at 37° C.

In all cases below wherein additional coating compositions (e.g. RS, zein protein, wax, and Ethylcellulose (ETHOCEL®) were applied onto the cores, fluidized bed air processor technology was utilized.

### Example 2

### Use of Fluidized Bed Air/N₂ Processor

Microcrystalline cellulose was fluidized in the warm atmosphere of the coating chamber. Next, probiotic microorganisms (Lactobacillus paracasei, Lactobacillus acidophilus, and Bifidobacteria bifidum) were dissolved in the different formulations then sprayed through a nozzle onto microcrystalline cellulose. The solvent or solvent mixtures were then evaporated by warm air or nitrogen gas, and the additional coating material was adhered to the particles.

Determination of the viability of encapsulated probiotic cells was performed by dissolving the samples in saline (0.85% NaCl) and spread plating onto MRS agar (Difco) plates, after appropriate 10-fold serial dilutions. Several hours later, viable cell count, determined after a 48-hour incubation under anaerobic conditions at 37° C., is depicted in Table 2. Viability of over or close to 70% was achieved in a number of samples.

Anaerobic jars and gas generating kits (Oxoid Ltd.) were used for creating anaerobic conditions. Plates containing 20-350 colonies were measured and recorded as colony forming units (cfu) per gram of the product or culture.

As depicted in Table 2, use of fluidized bed air/N₂ processor technology resulted in significant preservation of viability after drying in all samples.

TABLE 2
Survival of probiotic microorganisms during absorption and
drying onto microcrystalline cellulose.

| | Solids | | Bacterial | |
|---|---|---|---|---|
| | conc. | Suspension | count | Viability |
| Formulation | [% w/v] | Medium | [cell/gr] | [%]* |
| Maltodextrin DE19 | 30 | N₂ | 1.33 × 10⁸ | 31.7 ± 5^{#} |
| Maltodextrin | 30 | N₂ | 1.48 × 10⁸ | 34.5 ± 3 |
| DE19:Trehalose (2:1) | | | | |
| Maltodextrin | 50 | Air | 4.71 × 10⁷ | 18.5 ± 0.9 |
| DE19:Trehalose (3:2) | | | | |
| Maltodextrin | 20 | Air | 5.18 × 10⁸ | 35.9 ± 6.1 |
| DE3:Trehalose (1:1) | | | | |
| Maltodextrin | 20 | Air | 7.76 × 10⁸ | 24.9 ± 3.2 |
| DE5:Trehalose (1:1) | | | | |
| Maltodextrin | 20 | Air | 1.52 × 10⁹ | 38.2 ± 5.2 |
| DE19:Trehalose (1:1) | | | | |
| Maltodextrin | 20 | Air | 2.81 × 10⁹ | 35.3 ± 9.3 |
| DE19:Trehalose (1:1) | | | | |

*Expressed as mean ± error of the mean.

### Example 3

### Use of Ultrasonic Vacuum Spray Drying Process

Probiotic bacteria were dissolved in the different formulations prior to spray drying. Ultrasonic vacuum spray drying was performed as disclosed in U.S. Pat. No. 5,624,530 and described hereinabove in the specification. This method as well resulted in significant preservation of viability (Table 3), which was determined as described hereinabove in the previous section.

TABLE 3
Survival of probiotics during ultrasonic vacuum spray drying core formation.

| | Solids | | | Bacterial | |
|---|---|---|---|---|---|
| | conc. | | ^{#}Final | count | Viability, |
| Formulation | [% w/v] | Vacuum [torr] | a_{w} | [cell/gr] | [%] |
| Maltodextrin DE19 | 10 | 17 | 0.112 | 2.94 × 10⁷ | 20.4 ± 0.8 |
| Maltodextrin DE19 | 15 | 17 | 0.044 | 4.75 × 10⁷ | 18.2 ± 3.1 |
| Maltodextrin DE19 | 22 | 17 | 0.117 | 8.00 × 10⁷ | 29.2 ± 4.9 |
| Maltodextrin DE19 | 22 | 17 | 0.212 | 7.59 × 10⁷ | 27.7 ± 4.9 |
| Maltodextrin DE19 | 25 | 17 | 0.245 | 9.86 × 10⁷ | 38 ± 8.5 |
| Maltodextrin DE19 | 25 | 17 | 0.415 | 8.37 × 10⁷ | 32.2 ± 7.9 |
| Maltodextrin DE19 | 23 | 25 | 0.078 | 1.15 × 10⁸ | 25.2 ± 1.2 |
| Maltodextrin DE19:Trehalose | 20 | 17 | 0.108 | 1.48 × 10⁸ | 29.6 ± 4.0 |
| (1:1) | | | | | |
| Maltodextrin DE19:Trehalose | 30 | 17 | 0.214 | 1.15 × 10⁸ | 47.5 ± 6.9 |
| (1:1) | | | | | |
| Maltodextrin DE19:Trehalose | 30 | 17 | 0.490 | 6.64 × 10⁷ | 27.5 ± 4.4 |
| (1:1) | | | | | |
| Maltodextrin DE19:Trehalose | 27 | 25 | 0.125 | 1.98 × 10⁸ | 50.4 ± 13.4 |
| (2:1) | | | | | |
| Maltodextrin DE19:Trehalose | 27 | 25 | 0.168 | 1.64 × 10⁸ | 41.8 ± 7.5 |
| (2:1) | | | | | |
| Maltodextrin DE19:Trehalose | 29 | 17 | 0.186 | 1.56 × 10⁸ | 49.8 ± 7.3 |
| (2:1) | | | | | |
| Maltodextrin DE19:Trehalose | 29 | 17 | 0.438 | 6.87 × 10⁷ | 21.7 ± 8.3 |
| (2:1) | | | | | |
| Maltodextrin DE5 | 20 | 25 | 0.064 | 8.16 × 10⁷ | 39.5 ± 3.7 |
| Maltodextrin DE5 | 20 | 25 | 0.103 | 1.11 × 10⁸ | 53.4 ± 7.6 |
| Maltodextrin DE5 | 20 | 25 | 0.126 | 1.55 × 10⁸ | 56.9 ± 12.6 |
| Maltodextrin DE5 | 20 | 25 | 0.155 | 8.59 × 10⁷ | 41.5 ± 5.4 |
| Maltodextrin DE5:Trehalose | 20 | 25 | 0.129 | 3.18 × 10⁸ | 65.0 ± 10.4 |
| (1:1) | | | | | |
| Maltodextrin DE5:Trehalose | 20 | 25 | 0.210 | 2.39 × 10⁸ | 70.6 ± 6.2 |
| (1:1) | | | | | |
| Maltodextrin DE5:Trehalose | 30 | 25 | 0.282 | 3.36 × 10⁸ | 64.3 ± 4.1 |
| (1:1) | | | | | |
| Maltodextrin DE5:Trehalose | 28 | 25 | 0.249 | 3.31 × 10⁸ | 62.8 ± 7.9 |
| (2:1) | | | | | |

^{#}water activity

### Example 4

### Use of Spray Freezing—Freeze Drying Process

Probiotic bacteria were dissolved in the different formulations prior to spray freezing/freeze drying. A suspension of probiotic bacteria was sprayed by a nozzle or needle into liquid nitrogen. The frozen particles were further freeze dried by conventional freeze-drying equipment for 24-72 hours (depending on desired water activity of the product).

Determination of the viability of encapsulated probiotic cell samples was performed as described above. This method as well resulted in significant preservation of viability (Table 4).

TABLE 4
Survival during spray freezing - freeze drying core formation.

| | Solids | | Freeze | Bacterial | |
|---|---|---|---|---|---|
| | conc. | Spraying | drying shelf | count | Viability |
| Formulation | [% w/v] | device | temp. [° C.] | [cell/gr] | [%] |
| Maltodextrin DE19 | 20 | Needle | 20 | 3.59 × 10⁷ | 10.9 ± 2.2 |
| Maltodextrin DE19 | 20 | Needle | (−30) | 1.15 × 10⁸ | 34.8 ± 0.8 |
| Maltodextrin DE19 | 40 | Nozzle | (−30) | 3.84 × 10⁷ | 25.2 ± 3.7 |
| Maltodextrin DE19:Trehalose | 20 | Nozzle | (−30) | 2.05 × 10⁸ | 54.0 ± 6.8 |
| (1:1) | | | | | |
| Maltodextrin DE19:Trehalose | 30 | Nozzle | (−30) | 3.02 × 10⁸ | 65.8 ± 7.7 |
| (1:1) | | | | | |
| Maltodextrin DE19:Trehalose | 40 | Needle | 20 | 2.01 × 10⁸ | 45.2 ± 2.5 |
| (1:1) | | | | | |
| Maltodextrin DE19:Trehalose | 40 | Needle | (−30) | 2.10 × 10⁸ | 47.3 ± 1.7 |
| (1:1) | | | | | |
| Maltodextrin DE19:Trehalose | 40 | Nozzle | (−30) | 6.57 × 10⁸ | 41.2 ± 5.8 |
| (1:1) | | | | | |
| Maltodextrin DE19:Trehalose | 30 | Needle | 20 | 6.95 × 10⁷ | 21.3 ± 6.5 |
| (2:1) | | | | | |
| Maltodextrin DE19:Trehalose | 30 | Needle | (−30) | 2.25 × 10⁸ | 68.9 ± 2.4 |
| (2:1) | | | | | |
| Maltodextrin DE19:Trehalose | 30 | Nozzle | (−30) | 2.74 × 10⁸ | 57.1 ± 14.9 |
| (2:1) | | | | | |
| Maltodextrin DE5 | 20 | Needle | 20 | 6.23 × 10⁷ | 20.7 ± 2.3 |
| Maltodextrin DE5 | 20 | Needle | (−30) | 3.86 × 10⁷ | 12.8 ± 0.9 |
| Trehalose | 30 | Nozzle | 20 | 4.45E+07 | 37.3 ± 5.9 |
| Trehalose | 30 | Nozzle | (−30) | 5.80E+07 | 48.7 ± 7.4 |
| RS | 8 | Needle | 20 | 1.34 × 10⁷ | 1.0 ± 0.1 |
| RS | 8 | Needle | (−30) | 1.24 × 10⁸ | 9.3 ± 1.1 |
| RS:Trehalose (1:1) | 17 | Needle | 20 | 2.05 × 10⁸ | 32.8 ± 0.8 |
| RS:Trehalose (1:1) | 17 | Needle | (−30) | 1.94 × 10⁸ | 31.2 ± 2.2 |

### Example 5

### Application of Additional Coatings to the Microcapsules

Matrices containing probiotic microorganisms, containing DE3:Trehalose 1:1 and dried by air, were suspended in using fluidized bed air processor equipment and coated by several layers of wall materials (wax, ETHOCEL®, maltodextrin, resistant starch). Determination of the viability of the coated probiotic cells was performed by dissolving the samples in saline (0.85% NaCl) using a Stomacher® blender and spread plating on MRS agar (Difco) plates, after appropriate 10-fold serial dilutions. As depicted in Table 5, the encapsulation procedure enabled high viability of the encapsulated probiotics through the manufacturing process.

TABLE 5
Survival of Microencapsulated Probiotics during coating processes.

| Coatings (% w/w) | Core (% w/w) | Probiotics Survival % |
|---|---|---|
| 30% Wax | 70% | 99% |
| 25% Wax & 15% ETHOCEL ® | 60% | 66% |
| 24% Wax & 20% ETHOCEL ® | 56% | 61% |
| 21% Wax & 30% ETHOCEL ® | 49% | 49% |
| 30% MD15 | 70% | 82% |
| 20% Resistant Starch | 80% | 11% |

### Example 6

### Stability of Encapsulated Probiotics in Conditions Simulating Stomach Acidity

Matrices containing probiotic microorganisms were_suspended in a fluidized bed air processor apparatus and coated by several layers of wall materials (wax & ETHOCEL®). To test the stability of the encapsulated probiotics in stomach-like conditions, microcapsules were incubated for one hour in saline solution with pH=2, and 10 rpm shaking. Determination of the viability of the coated probiotic cells was performed by dissolving the samples in saline (0.85% NaCl) using a Stomacher blender, and spread plating on MRS agar plates (Difco), after appropriate 10-fold serial dilutions. Survival of encapsulated bacteria exceeded that of the uncoated bacteria by 1600 to 2600 fold.

### Example 7

### Evaluation of Shelf Life

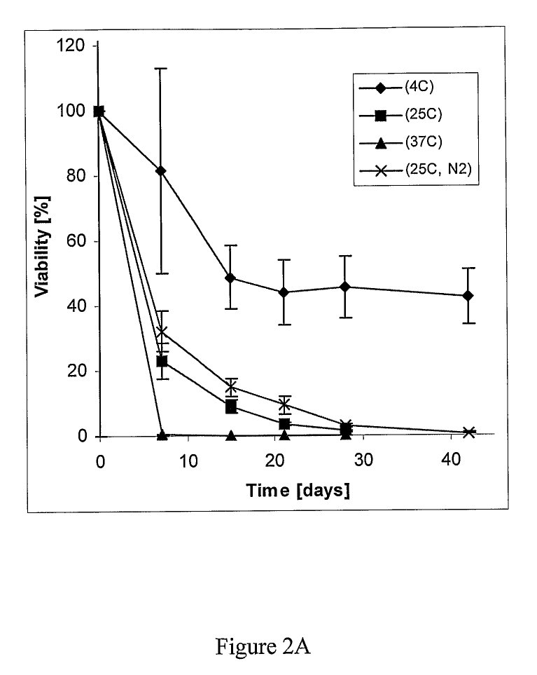

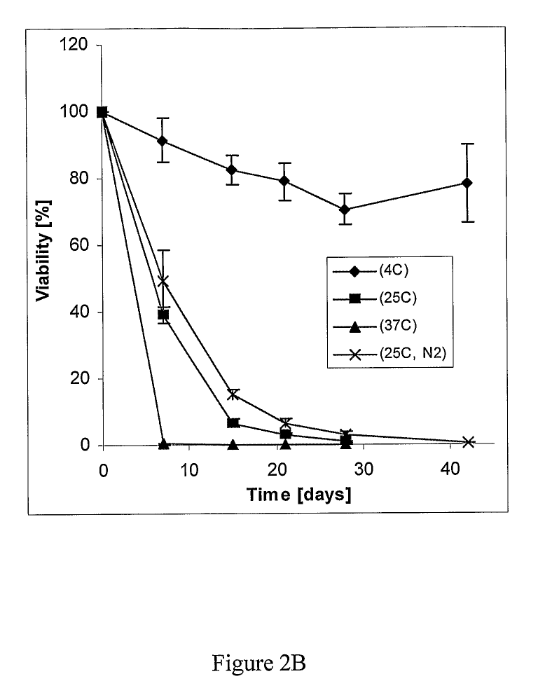

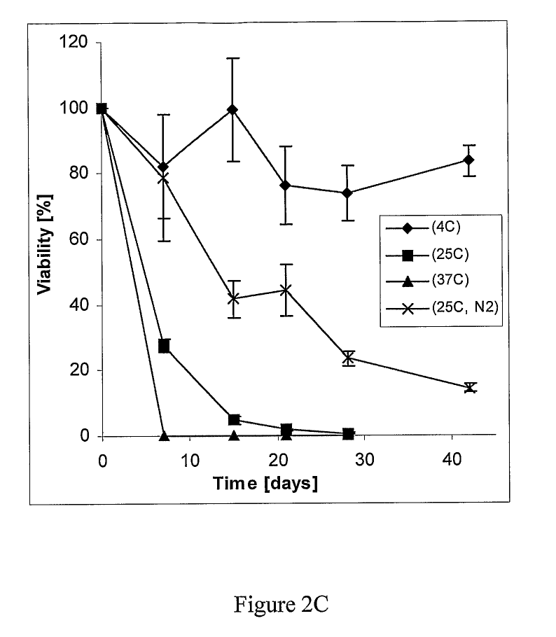

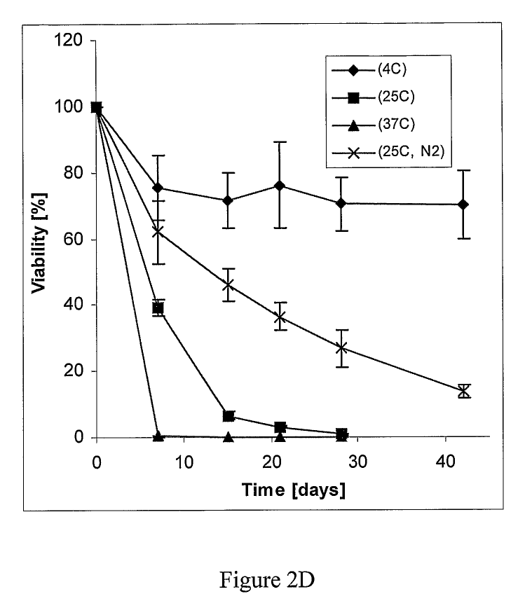

Four specific formulations were selected for exemplifying the viability of the probiotics within the composition of the invention during storage: Maltodextrin DE5 (FIG. 2A); Maltodextrin DE19 (FIG. 2B); Maltodextrin DE5: Trehalose (1:1) (FIG. 2C); and Maltodextrin DE19:Trehalose (1:1) (FIG. 2D), as well as coated capsules. Encapsulated probiotics were stored at three different temperatures, 4° C. (air), 25° C. (air and N₂) and 37° C. (air).

Ultrasonic vacuum spray drying was used to manufacture formulations with the following percentages of solids: Maltodextrins—20% w/v (FIG. 2 A-B), maltodextrin:trehalose (1:1)—30% w/v (FIG. 2 C-D).

As depicted in FIG. 2, viability for the majority of the compositions was above 70% after 43 days of storage at 4° C.

### Example 8

### Incorporation of Microcapsules into a Confectionary Product

Probiotic strains of Lactobacilli and Bifidobacteria were adsorbed on Microcrystalline cellulose using bacterial dispersion in Maltodextrin DE6: Trehalose (1:1). Subsequently, the microcapsules were coated with Wax—30% w/w, and then Ethylcellulose—15% w/w. The microencapsulated probiotics were added to the mix of a confectionary product prior to its forming. Determination of the viability of the coated probiotic cells was performed by dissolving the samples in saline (0.85% NaCl) using a Stomacher apparatus, and spread plating on MRS agar (Difco) plates, after appropriate 10-fold serial dilutions. Probiotic bacteria counts showed that the final probiotics content exceeded 10̂7 cfu/gr, which is required for defining the product as probiotic.

| Sample | Microcapsules in product [%] | Final probiotic content [CFU/gr] |
|---|---|---|
| A | 5% | 4.15E+07 |
| B | 2% | 1.55E+07 |
| C | 5% | 3.63E+07 |

It is to be understood that, while the invention has been described in conjunction with the preferred specific embodiments thereof, the foregoing description is intended to illustrate and not limit the scope of the invention. Other aspects, advantages, and modifications will be apparent to those skilled in the art to which the invention pertains. Furthermore, the practice of the present invention will employ, unless otherwise indicated, conventional techniques of microcapsule and coating formulation that are within the skill of the art. Such techniques are fully explained in the literature.

1.-51. (canceled)

52. A microcapsule comprising (a) a core, the core comprising (i) a probiotic microorganism; (ii) a dextrin; and (iii) a cytoprotective disaccharide or oligosaccharide; the core being in the form of a glassy matrix, and (b) a moisture-resistant coating.

53. The microcapsule of claim 52, wherein the probiotic microorganism is selected from a Lactobacillus and a Bifidobacterium.

54. The microcapsule of claim 52, wherein the dextrin is a maltodextrin.

55. The microcapsule of claim 52, wherein the cytoprotective disaccharide is trehalose.

56. The microcapsule of claim 52, wherein the weight ratio between the dextrin and the cytoprotective disaccharide or oligosaccharide is within the range of 0.5:1 to 3:1.

57. The microcapsule of claim 53, wherein the weight ratio between the bacteria and the other components of the core is within the range of 0.5% to 30%.

58. The microcapsule of claim 52, wherein the core has a water activity below 0.4.

59. The microcapsule of claim 52, wherein the core further comprises a microcrystalline cellulose, spray-dried starch, or spray-dried resistant starch.

60. The microcapsule of claim 59, wherein the mass of the microcrystalline cellulose, spray-dried starch, or spray-dried resistant starch is within the range of 60 to 90% of the total mass of the core.

61. The microcapsule of claim 52, wherein the microcapsule is coated with a food-grade coating.

62. The microcapsule of claim 61, wherein the food-grade coating comprises a substance selected from the group consisting of: wax, shellac, resistant starch, zein protein, ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate

63. The microcapsule of claim 61, wherein the food-grade coating is an enteric coating.

64. A probiotic composition comprising the microcapsule of claim 52.

65. A dry food product comprising an edible material and the microcapsule of claim 52.

66. A glassy matrix comprising a maltodextrin, a cytoprotective disaccharide or oligosaccharide, and a probiotic microorganism selected from a Lactobacillus and a Bifidobacterium.

67. The glassy matrix of claim 66, wherein the maltodextrin has a dextrose equivalent within the range of 2 to 30.

68. The glassy matrix of claim 66, wherein the cytoprotective disaccharide or oligosaccharide is trehalose.

69. The glassy matrix of claim 66, wherein the glassy matrix has a water activity below 0.4.

70. The glassy matrix of claim 66, further comprising a moisture-resistant coating.

71. The glassy matrix of claim 66, wherein the weight ratio between the maltodextrin and the cytoprotective disaccharide or oligosaccharide is within the range of 0.5:1 to 3:1.

72. The glassy matrix of claim 66, wherein the weight ratio between the bacteria and the other components of the glassy matrix is within the range of 0.5% to 30%.

73. The glassy matrix of claim 66, wherein the glassy matrix further comprises a microcrystalline cellulose, spray-dried starch, or spray-dried resistant starch.

74. The glassy matrix of claim 73, wherein the mass of the microcrystalline cellulose, spray-dried starch, or spray-dried resistant starch is within the range of 60 to 90% of the total mass of the glassy matrix.

75. A dry food product comprising an edible material and the glassy matrix of claim 66.

76. A dry food product comprising an edible material and the glassy matrix of claim 70.
